(19) United States
(12) Patent Application Publication
(10) Pub. No.: US 2007/0092865 A1
(43) Pub. Date: Apr. 26, 2007

(54) Detection and characterization of psychoactives using parallel multi-site assays in brain tissue

(76) Inventors: Gary Lynch, Irvine, CA (US); Laura L. Colgin, Irvine, CA (US); Rafael H. Saavedra, Newport Beach, CA (US)

Correspondence Address: MORRISON & FOERSTER LLP, 755 PAGE MILL RD, PALO ALTO, CA, 94304-1018, US

(21) Appl. No.: 11/600,958

(22) Filed: Nov. 15, 2006

Related U.S. Application Data
(62) Division of application No. 10/271,694, filed on Oct. 15, 2002.
(60) Provisional application No. 60/329,011, filed on Oct. 12, 2001.

(51) Int. Cl. C12Q 1/00; G06F 19/00

(52) U.S. Cl. 435/4; 702/19

(57) ABSTRACT

This invention relates to methods and devices for the detection and characterization of psychoactive compounds by analyzing alterations of network level physiological characteristics before and after the introduction of a candidate sample onto an in vitro neuronal tissue sample. The invention further provides a software package that enables an operator to deliver a timed electrical pulse to neuronal samples at a specific point in their spontaneous or induced oscillations. Such temporal stimulations trigger unexpected and useful network level physiological responses.

### FIELD OF THE INVENTION

The present invention relates to a method and device for the detection and characterization of psychoactive compounds. Specifically, the detection and characterization of psychoactive compounds using network level responses in neuronal tissue samples is described.

### BACKGROUND OF THE INVENTION

The great majority of synapses in the hippocampus arise from associational and cortical afferents that use glutamate as a transmitter. As with other telencephalic areas, the hippocampus also receives significant projections from several subcortical structures that utilize an array of transmitters other than glutamate. The largest and best studied of the subcortical projections to the hippocampus is the cholinergic input from the medial septum/diagonal bands. These afferents generate the 4-7 Hz theta rhythm by mechanisms that are now fairly well understood (Vertes et al., Neuroscience 81: 893-926 (1997)). Much less is known about how the cholinergic inputs modulate hippocampal responses to activation of glutamatergic pathways.

While several studies have shown that infusion of cholinergic agonists into hippocampal slices causes the near immediate appearance of rhythmic oscillations, there is disagreement regarding the dominant frequency of the activity. Theta, beta (13-30 Hz), and gamma (˜40 Hz) rhythms have each been reported to be triggered by application of carbachol (Konopacki et al., Brain Res 405: 196-198 (1987); Huerta et al., Nature 364: 723-725 (1993); Williams et al., J Neurophysiol 78: 2631-2640 (1997); Fisahn et al., Nature 394: 186-189 (1998); Fellous et al., Hippocampus 10: 187-197 (2000); Shimono et al., J Neurosci 20: 8462-8473 (2000)). Recent work using multi-electrode recording techniques has found that carbachol ellicits regionally discrete beta activity, sometimes accompanied by gamma waves, in the majority of slices. Two dimensional Current Source Density analyses suggests that bursts of pyramidal cell discharges, spread of excitation through collateral projections, and activation of apically-directed feedback interneurons generates the beta waves (Shimono et al., supra). These ideas are in broad agreement with conclusions drawn from single cell studies of carbachol's actions (Nakajima et al., Proc Natl Acad Sci USA 83: 3022-3026 (1986); Madison et al., J Neurosci 7: 733-741 (1987); Behrends et al., J Neurophysiol 69: 626-629 (1993)) and from recent work on the distribution of muscarinic receptors in hippocampus (Levey et al., J Neurosci 15: 4077-4092 (1995); Hajos et al., Neuroscience 82: 355-376 (1998)).

Consequently, there has been considerable effort to develop methods and devices for the characterization and detection of psychoactive compounds using carbachol-induced rhythmic oscillations. A problem encountered in such efforts has been that at low concentrations, many psychoactive compounds have a relatively small probability of changing the activity of single neurons or even small groups of neurons (e.g., currents, firing rate). Although there may be compound-induced changes at such levels of observation, the changes may be too weak to detect and/or such changes may occur with low probability. When the activity of many neurons is synchronized, as with cortical rhythms, individual cells are acting together as a system. This characteristic serves to amplify the small probabilities of functional changes at the single cell level, producing a higher probability of detectable changes. This serves to lower the threshold concentration at which agents can be detected, e.g., by using a network-based screening device. Similarly, the thresholds can be brought closer to concentrations known to produce cognitive and behavioral effects. However, many in vivo or in vitro models lack some or all of these important features.

None of the cited documents discuss assay systems that can produce the enhanced diagnostic characteristics, and improved detection attributes, mentioned above, and new ways to discover, investigate, characterize, and develop psychoactive compounds.

### SUMMARY OF THE INVENTION

The present invention provides methods and devices for the detection and characterization of psychoactive compounds by analyzing network level responses in in vitro neuronal tissue samples.

In one variation, the method and device involve capturing (measuring) at least one spontaneous oscillation from the in vitro neuronal tissue sample. Voltage peaks and troughs of the oscillation are then determined, and at least one timed electrical pulse is delivered at a specific point in the oscillation to produce a network level electrical baseline.

In another variation, induced oscillations instead of spontaneous oscillations are captured and subjected to at least one timed electrical pulse to produce a network level electrical baseline. The oscillations may be induced by chemical compositions, co-deposited neuronal tissue, or electrical stimulations. The chemical compositions typically mimic the actions of acetylcholine, serotonin, or a catecholamine. In a preferred variation, the chemical composition includes carbachol. The chemical composition is usually a stimulating composition.

Once a network level electrical baseline is obtained, the in vitro neuronal tissue sample is contacted with a candidate sample composition, and a network level electrical response is measured. The network level electrical baseline and network level electrical response is then compared to detect the presence or absence of a psychoactive compound in the candidate sample composition and to characterize the candidate sample composition.

The various oscillations are typically those found in extracellular voltage. For instance, they may be a theta, beta, or gamma EEG waves. The network level electrical baselines and network level electrical responses are typically comprised of extracellular voltages. For example, they may be 100 microvolts for the slow, negative-going potential or 50 microvolts for the initial, fast negative-going potential.

It is also desirable to use a multi-electrode dish (“MED”) to measure individual oscillations or network level baselines and network level responses so that a number of different active or less active sites on the neuronal sample may be simultaneously or sequentially sampled. Use of the MED permits measurement and calculation of spatial relationships; both measured and calculated, amongst the values of the neural oscillations. The multi-electrode nature of the MED also enables the determination and characterization of region-specific effects within the given in vitro neuronal sample.

Appropriate mathematical analysis of the oscillations of extracellular voltage can include a Fast Fourier Transform (FFT) of oscillations measured at a single spatial point to enhance differences in amplitude and frequency of the before-and-after single-site measurements. Similarly, the sequence of oscillations of extracellular voltage obtained in an array as a function of time may be subjected to Current Source Density (CSD) analysis to produce and depict current flow patterns within the in vitro neuronal tissue sample. Additionally, the network level responses can be analyzed by separating the waveforms into fast and slow components and calculating local maxima and minima, decay time, and the like.

Another portion of the method includes: 1) the use of tissue preparation methods that preserve network structure, 2) electrical stimulation patterns that tend to stimulate or induce a network level, widespread neuronal response, characterized by sustained time courses and distributed activity of neurons across an entire network.

Yet another portion of the method includes the in vitro measurement of muscle electrical activity. Muscle, in the same manner as neuronal tissue, exhibits spontaneous electrical waveforms and is “excitable.” Changes in the electrical activity pattern of muscle, e.g., smooth muscle, thus may also be used to detect and characterize candidate sample compositions, similar to the processes and methods herein described for in vitro neuronal tissue samples.

### BRIEF DESCRIPTION OF THE DRAWINGS

The accompanying drawings illustrate and explain the principles of the invention. They are not intended to limit the scope of the invention in any way.

FIG. 1-1 is a flowchart of the control system, which enables an operator to stimulate neuronal tissue slices exhibiting oscillatory responses at a particular time relative to their fundamental oscillation activity.

FIG. 1-2 shows the algorithm for the Stimulation Control System: A) captured spontaneous response; B) hi-cut filtered spontaneous response; C) detected positive and negative peaks (false peaks are circled); D) elimination of false peaks; E) histogram of the positive and negative peaks; and F) cumulative probability distribution for the positive and negative peaks.

FIG. 1-3 demonstrates the effect of the Stimulation Control System: A) stimulation delivered at time 500 ms without using this system, and capture of five consecutive responses; B) delivery of stimulation at the positive peak and capture of five consecutive responses; and C) delivery of stimulation at the negative peak (trough) and capture of five consecutive responses.

FIG. 2 depicts carbachol-induced beta rhythms in hippocampal tissue: A) hippocampal slice placed upon a medium array of 64 electrodes (interelectrode spacing: 300 μm); B) spontaneous activity prior to carbachol infusion; single unit activity was detectable, but no synchronized cell firing was present; C) Fast Fourier transforms of beta rhythms; normalized power spectra computed for all 64 electrodes following infusion of 25 μM carbachol showed that the dominant frequency was in the beta range and power was maximal in the apical dendritic field; and D) beta rhythms recorded after carbachol infusion; the waveform of the rhythms reversed across the cell body layer (e.g., electrode C2 vs. C4), indicting that activity was locally generated; amplitudes were large in the apical dendrites (e.g., electrode C4) and directly on the cell bodies (e.g., electrode E3). Rhythms remained stable for over two hours. Calibration bars: 250 ms, 100 μV.

FIG. 3 depicts evoked responses throughout the hippocampal network following stimulation of Schaffer collaterals in the presence and absence of carbachol: A) hippocampal slice placed upon medium array of electrodes (interelectrode spacing: 300 μm); electrode F3 was chosen for S-C stimulation; B) evoked potentials across all 64 sites in the control condition; note that responses did not propagate throughout the entire network; activity was limited to the apical dendritic fields of CA3 (e.g., electrode F4) and CA1 (e.g., electrode E3); phase reversals were prominent across the cell body layer in CA1 (e.g., electrodes E1 vs. E3); C) evoked potentials in the presence of carbachol in which a complex response was not generated; responses and regional distribution were similar to the control condition; background beta rhythm activity resumed almost immediately following stimulation; and D) evoked response in the presence of beta waves in which a complex, reverberating response was generated; initial fast negative-going potentials were observed in the apical dendritic fields of CA3 and CA1; however, instead of a prompt return to rhythmic activity, a sustained response was observed. High-frequency cell spiking was recorded from CA3 pyramidal cells (e.g., electrode G5), presumably driven by an associated negative-going waveform in the basal dendritic field of CA3 (e.g., electrode E6). The apical dendritic field of CA3 exhibited a slow positive-going potential followed by a slow negative-going potential. These apical-basal slow potential phase relationships were reversed in CA1. A delayed negative-going slow potential was recorded in the apical dendritic field of CA1 (e.g., electrode E3) with a corresponding delayed positive-going slow potential in the basal dendritic field (e.g., electrode El). Note the increased spread of activation across the entire network during this complex response. Calibration bars: 100 ms, 200 μV. Stimulation artifacts appear as a vertical line at far left of each trace.

FIG. 4 demonstrates evoked potentials in the CA1 region of hippocampus following stimulation to the Schaffer collateral pathway in the presence (black) and absence (gray) of carbachol-induced beta waves. The horizontal, dotted line in the center of each trace denotes 0 μV. Stimulation in the absence of any rhythmic activity resulted in a stereotyped response, consisting of a fast, negative-going potential followed by a positive-going after-potential in CA1 stratum radiatum (bottom). The phase reversal of the response was recorded from CA1 stratum oriens (top). The entire event was finished by 40 ms post-stimulation. In the presence of cholinergically-induced rhythms, a markedly different response was recorded. In the apical dendritic field of CA1 (bottom), the primary fast response was followed by a sequence of slow potentials. The first was negative-going and was associated with high-frequency spiking. The second slow potential was positive-going and did not return to baseline until 140 ms after stimulation. The response in CA1 stratum radiatum was phase-reversed in stratum oriens, such that a positive-going slow potential was followed by a negative-going slow potential and a return to baseline at 140 ms post-stimulation.

FIG. 5 depicts evoked potentials in field CA3 region of the hippocampus in the presence (black) and absence (gray) of carbachol-driven beta rhythms following stimulation to the Schaffer collateral pathway. The dotted horizontal line in each trace indicates 0 μV. In the basal dendritic field of CA3 (top left), the control response was negligible, as was the fast component of the carbachol response. The initial phase of the slow potential of the carbachol response was a negative-going waveform with high-frequency spiking visible. The negative-going slow potential was followed by a positive-going potential, which returned to baseline at approximately 125 ms. In the apical dendrites (bottom left), a typical control response was recorded, consisting of a fast negative-going waveform followed by an after-hyperpolarizing potential. In the presence of carbachol, the apical dendritic response resembled the control response for a short time (<5 ms post-stimulation) before veering off into a positive-going waveform (˜10 ms post-stimulation). Again, high-frequency spikes were visible during this phase. Note that the apical response was a phase-reversal of the basal response, such that the initial positive-going waveform was followed by a negative-going waveform. The source of the high-frequency spiking was the CA3 pyramidal cells (right traces, top and bottom), and spiking was observed across the entire extent of CA3 stratum pyramidale. Note that control responses in the cell body were insignificant. The negative-going waveform in the CA3 basal dendritic field was likely driving the high-frequency firing of CA3 pyramidal cells through the dense associational system of CA3.

FIG. 6 shows high frequency bursting during a complex response. A single stimulation pulse was delivered to the S-C pathway in the presence of 25 μM carbachol. Responses recorded from the hippocampal slice depicted in the left panel were high pass filtered at 100 Hz to remove the slow potentials during the time segment from 10 to 80 ms after stimulation (right panel). It was evident that bursting was most prominent in field CA3, especially in stratum pyramidale. Calibration bars: 50 ms, 0.5 mV.

FIG. 7 demonstrates two-dimensional current source density estimates for evoked responses in the presence (top) and absence (bottom) of carbachol-induced rhythms. Depolarizing current sinks are depicted in gray with hyperpolarizing current sources shown in white. The outline of the pyramidal cell bodies is depicted as white triangles, with larger cell bodies delineating field CA3 and smaller cell bodies in CA1. At 4 ms after stimulation to the S-C pathway, differences between control and carbachol responses were minimal. At 10 ms post-stimulation during the carbachol response, a large current source appeared in the apical dendrites of CA3, as a current sink appeared in the basal dendrites, especially apparent in CA3c. In the control response, activity did not spread across the entirety of CA3 and the CA3 apical source/basal sink dipole was not present. At 23 ms, the CA3 cell bodies and basal dendritic fields continued to be dominated by current sinks, while the CA3 apical source also remained. A prolonged depolarizing current sink was observed in the apical dendritic field of CA1, with a corresponding current source in CA1 stratum oriens. The control response, on the other hand, displayed weak activation at this time, with an after-hyperpolarization in the CA1 apical dendrites as the only distinguishing feature. This weak hyperpolarizing current source lingered in the CA1 apical dendrites of the control response at 30 ms, and stood in contrast to the strong depolarizing current sink observed in the same location at the same time point during the carbachol response.

FIG. 8 shows a network level evoked response to single pulse Schaffer commissural stimulation changes upon infusion of the biohazard compound, heptachlor.

FIG. 9 shows a network level evoked response to single pulse Schaffer commissural stimulation changes upon infusion of the ampakine CX1036.

FIG. 10 shows a network level evoked response to single pulse Schaffer commissural stimulation changes upon infusion of the ampakine CX554. Calibration bars are 50 msec and 0.4 mV.

FIG. 11 shows a network level evoked response to single pulse Schaffer commissural stimulation changes upon infusion of the ampakine CX682.

### DETAILED DESCRIPTION

Recited is process and device for the detection and/or characterization of psychoactive compounds using rhythmic oscillations of extracellular voltage (potential) in in vitro neuronal tissue samples. The oscillations are generally stimulated or induced, resulting in a “network-level response”, by using certain electrical stimulation techniques that are discussed in more detail below.

The measurement of electrical waveforms in in vitro neuronal tissue over the spatial array of a neuronal sample may be found in the various descriptions of such devices found in U.S. Pat. Nos. 5,563,067 and 5,810,725 to Sugihara et al., the entirety of which are incorporated by reference. Additional details relating to the devices, methods, and processes herein described for the present invention may also be found in U.S. patent application Ser. No. 09/602,629 which is herein incorporated by reference in its entirety.

Definitions

As used herein, the term “sink” refers to current being absorbed from the extracellular medium into a neuronal element.

As used herein, the term “source” refers to current being injected into the extracellular medium from within a neuronal element. Or, in other words, the current is “sourced by” the neuronal element, i.e., derived from the neuronal element and then transferred to the extracellular medium.

As used herein, the term “hippocampus” refers to a region of the telencephalon that is located behind the temporal lobes and has been implicated in memory formation and retrieval in humans and other animals.

As used herein, the term “hippocampal slice” refers to a physical slice of hippocampal tissue that is approximately 100-500 micrometers in thickness that can be used on the electrophysiological recording apparatus described herein.

As used herein, the term “CA1”, “CA2”, “CA3”, and “CA4” refer to one of four regions of hippocampus.

As used herein, the term “dendrites” refers to the highly branched structure emanating from the cell body of the nerve cells.

As used herein, the term “network level” refers to a systems level observation; for example, groups of cells acting simultaneously as opposed to the isolated behaviors and characteristics exhibited by a single cell.

As used herein, the terms “network level response”, “network level electrical response”, and “network level evoked response” are used interchangeably and refer to a polysynaptic response involving groups of neurons and/or a polysynaptic response that incorporates groups of neurons after exposure to a candidate sample composition.

The terms “network level baseline” and “network level electrical baseline” are used interchangeably and refer to spontaneous or induced oscillations from neuronal samples that have been subjected to at least one timed electrical pulse.

As used herein, the terms “Schaffer collateral” and/or “Schaffer commissural” refer to the axonal pathway connecting CA3 and CA1 pyramidal cells.

Measuring Apparatus

In one variation, the inventive process uses a cell potential measuring electrode array that includes a plurality of measurement microelectrodes on a measuring region of an insulating substrate, a conductive pattern for connecting the microelectrodes to some region out of the microelectrode area, electric contacts connected to the end of the conductive pattern, an insulating film covering the surface of the conductive pattern, and a wall enclosing the region including the microelectrodes on the surface of the insulating film.

The array also includes a plurality of reference electrodes that may have comparatively lower impedance than the impedance of the measuring microelectrodes. The reference electrodes may be placed at various positions in the region enclosed by the wall and often at a specific distance from the microelectrodes. Furthermore, the electric contacts are usually connected between the conductive pattern for wiring of each reference electrode and the end of the conductive pattern. The surface of the conductive pattern for wiring of the reference electrodes is usually covered with an insulating film.

Typically, the microelectrodes are situated in a matrix arrangement in a rectangle having sides of, for example, 0.8 to 2.2 mm (in the case of 300 micrometer microelectrode pitch) or 0.8 to 3.3 mm (in the case of 450 micrometer microelectrode pitch). Four reference electrodes are situated at four corners of a rectangle of 5 to 15 mm on one side. More preferably, 64 microelectrodes are situated in eight rows and eight columns at central pitches of about 100 to 450 micrometers, preferably 100 to 300 micrometers. Preferably the microelectrodes and the reference electrodes are formed of layers of nickel plating, gold plating, and platinum black on an indium-tin oxide (ITO) film.

The insulating substrate (e.g., a glass substrate) may be nearly square. Plural electric contacts may be connected to the end of the conductive pattern and preferably are placed on the four sides of the insulating substrate. As a result, the layout of wiring patterns of multiple microelectrodes and reference electrodes is rather simple. Because the pitches of electric contacts may be made to be relatively large, electric connection through the electric contacts with external units is also simple.

The microelectrode region is usually very small. When observing the sample through a microscope, it is hard to distinguish position in both vertical and lateral directions. It is desirable to place indexing micro-marks near the microelectrode region to allow visual recognition through the microscope variously of direction, axes, and position.

It is even more preferable to perform the following sequence of events to determine electrode positions versus the anatomical correlates of the in vitro neuronal samples: 1) placing a control in vitro neuronal sample on the array in order that the array can cover the important area of the sample; 2) taking a picture of the control sample on the array; 3) recording the electrical activity from the control sample; 4) placing a test sample on the array in the same relative position as the control sample as accurately as possible; 5) taking a picture of the test sample on the array; 6) recording the electrical activity from the test sample; 7) comparing the control picture and the test picture; and 8) comparing the electrical activity from the control and test samples.

An alternative method is to use an object recognition algorithm (where the object is the gross anatomical structure of the in vitro neuronal sample) to compare object recognition algorithm data, and compare the electrical activity from the control and test samples.

In another variation, the cell potential measuring apparatus is made up of a cell placement device having cell potential measuring electrodes, contact sites for contacting with an electric contact, and an electrode holder for fixing the insulating substrate by sandwiching from above and beneath. The cell potential measuring electrodes may be connected electrically to the cell placement assembly device to allow processing of the voltage or potential signals generated by the sample and measured between each such microelectrode and the reference electrodes. The cell potential measuring assembly may include a region enclosed by a wall for cultivating sample neuronal cells or tissues. It may also optionally include an optical device for magnifying and observing optically the cells or tissues cultivated in the region enclosed by the wall. This cell potential measuring apparatus may also further include an image memory device for storing the magnified image obtained by the optical device.

In general, a personal computer having installed measurement software is included to accept the measured cell potentials. The computer and cell placement device are typically connected through an I/O board for measurement. The I/O board includes an A/D converter and a D/A converter. The A/D converter is usually for measuring and converting the resulting potentials; the D/A converter is for sending stimulus signals to the sample, when needed.

The measurement software installed in the computer may include software for setting conditions for giving a stimulus signal, forming the stimulus signal, and for processing and recording the obtained detection signal from the neuronal cells or tissue slices. The computer may also control any optical observation devices (e.g., SIT camera or image memory device) and the cell culture system.

In one variation, the extracellular potential detected from the neuronal tissue sample may be displayed in real time. In another variation, the recorded spontaneous electrical activity and induced potential is displayed by overlaying the waveform recordings on the microscope image of the cell. Alternative variations include software with image processing capabilities, e.g., feature recognition, edge detection, edge enhancement, or algorithmic capabilities. When measuring the potential, the entire recorded waveform is usually displayed visually and then correlated to the position of the waveform in the neuronal tissue sample.

Concerning data analysis or processing, applications such as Fast Fourier Transform (FFT) analysis, coherence analysis, and correlation analysis may be used. In the variation discussed below, Current-Source Density Analysis (CSD) is employed. Other useable functions may include single spike separation function using waveform discrimination, temporal profile display function, and topography display function. Other functions may also include various multivariate signal processing techniques, e.g., time series modeling. These analysis results may be displayed by overlaying on the displayed images of the neuronal sample stored in the image memory device.

When a stimulus signal is issued from the computer, this stimulus signal is sent to the cell placement device through a D/A converter and an isolator. The cell placement device includes a cell potential measuring electrode that may be formed, e.g., of 64 microelectrodes on a glass substrate in a matrix form and having an enclosing wall for maintaining the neuronal sample (e.g., cells or tissue slices) in contact with the microelectrodes and their culture fluid. Preferably, the stimulus signal sent to the cell placement device is applied to arbitrary electrodes out of the 64 microelectrodes and then to the sample or samples.

The induced (evoked) or spontaneous potential occurring between each microelectrode and reference potential (which is at the potential of the culture fluid) is passed through a 64-channel high sensitivity amplifier and an A/D converter into the computer. The amplification factor of the amplifier may be, e.g., about 80-100 dB, for example, in a frequency band of about 0.1 to 10 kHz, or to 20 Hz. However, when measuring the potential induced by a stimulus signal, by using a low-cut filter, the frequency band is preferably 1 Hz to 20 kHz.

In another variation, the apparatus may include a cell culture system having a temperature controller, a culture fluid circulation device, and a feeder for supplying, e.g., a mixed gas of air and carbon dioxide. The cell culture system may be made up of a commercial microincubator, a temperature controller, and CO₂ cylinder. The microincubator can be used to control in a temperature range of 0° C. to 50° C. by means of a Peltier element and is applicable to the liquid feed rate of 3.0 ml/min or less and gas flow rate of 1.0 liter/min or less. Or, alternatively, a microincubator incorporating a temperature controller may be used.

Data Measurement and Analysis

In general, the processes and methods described herein include simultaneous measurement and recording of the electrical activity of neuronal samples both spatially and temporally at each of the measurement sites. Additionally, they include observing the frequency and amplitude of the signals at each of the measurement sites in the spatial array. Furthermore, the processes and methods include viewing the placement and inherent physical boundaries of the neuronal tissue sample (margins correlating with the position of the sensors) using such instruments as optical devices, electronic sensing devices, or other devices which may be appreciated by one of skill in the art.

In use, the neuronal sample is placed upon the in vitro cell potential measuring electrode array and procedures that would be known to one skilled in the art are used for maintaining its viability during the testing. The neuronal sample may be cultured, if desired. Typical procedures are discussed below with respect to the Examples. Each of the microelectrodes is monitored, both as a function of time and as a function of frequency, for rhythmic oscillations of extracellular voltages or potentials, and for responses triggered by pulses and/or from the induction of psychoactive material. This produces an array of frequency and amplitude signals as a function of time. It is preferable to measure the oscillations from a region of near DC at 2 Hz to a region above 35 Hz. This permits measurement retention of the typical three frequency bands found in neuronal rhythmic activity: 1) 4 to 20 Hz (theta EEG); 2) 15 to 25 Hz (beta EEG); and 3) above 30 Hz (gamma EEG). The higher frequency band of 10 to 50 Hz, is also significantly instructive.

When induced or stimulated oscillations are desired, we have found it desirable to induce or stimulate these oscillations of extracellular voltage or potential variously by chemical, physiological, or anatomical methods. In one variation, neuronal tissue is contacted with a chemical composition including, e.g., one or more compounds that facilitate or mimic the actions of acetylcholine, serotonin, or catecholamines; however, contact with other compositions are acceptable. In another variation, the chemical composition includes one or more cholinomimetic compounds, e.g., carbachol (carbyl choline chloride).

We have also found it desirable to induce or stimulate network level neuronal responses by triggering the oscillations of extracellular voltage or potential using various physiological stimulation patterns. In one variation, physiological stimulation to localized regions of the tissue sample is used, e.g., to perforant path, mossy fiber, or Schaffer commissural regions.

In yet another variation, the inventive process includes determining, through the use of a predictive stimulation control system, exactly when such a stimulation pattern should be delivered to the tissue oscillations; for example, five 100 microsecond pulses delivered during the rising phase of slower ongoing oscillations. In the absence of oscillating activity, the exact time of stimulation delivery to the neuronal samples is not critical. However, when neuronal samples exhibit oscillatory behavior, the exact time of stimulation delivery relative to the fundamental oscillatory frequency may be significant because the delivered train of electrical pulses typically affects the future electrical behavior of the neuronal sample. The ability to control the exact time when a train of electrical pulses is delivered relative to the oscillatory behavior of the sample thus significantly enhances the discriminative power of the method. Furthermore, the process of monitoring, analysis, and predictions is preferably carried out continuously in order to guarantee that future pulses will synchronize with previous ones. It is particularly desirable to carry out the whole process in real time.

The underlying concept of the stimulation control system generally follows the following algorithm: 1) a spontaneous oscillation(s) from the neuronal sample is identified within a noisy signal and captured; 2) the fundamental frequency and phase of the oscillaton is determined; 3) future behavior of the oscillation is predicted in order to synchronize the delivery of a timed electrical pulse or a train of electrical pulses; and 4) the electrical pulse or train of electrical pulses is triggered and delivered at the appropriate time to obtain a network level electrical baseline. A candidate sample composition may then be added to the in vitro neuronal tissue sample, and the resulting oscillations measured to obtain a network level electrical response.

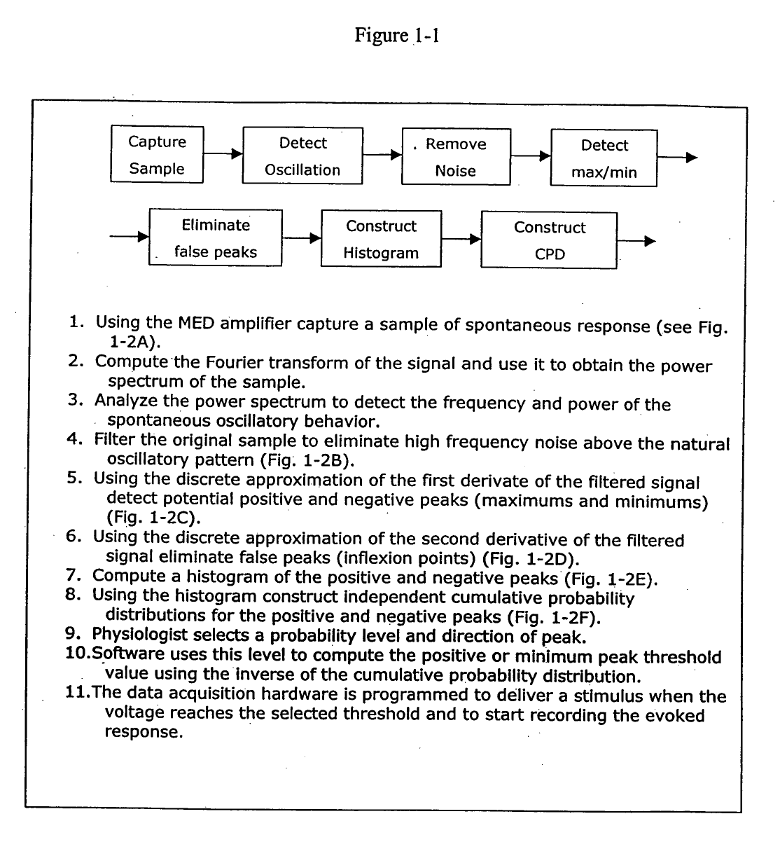

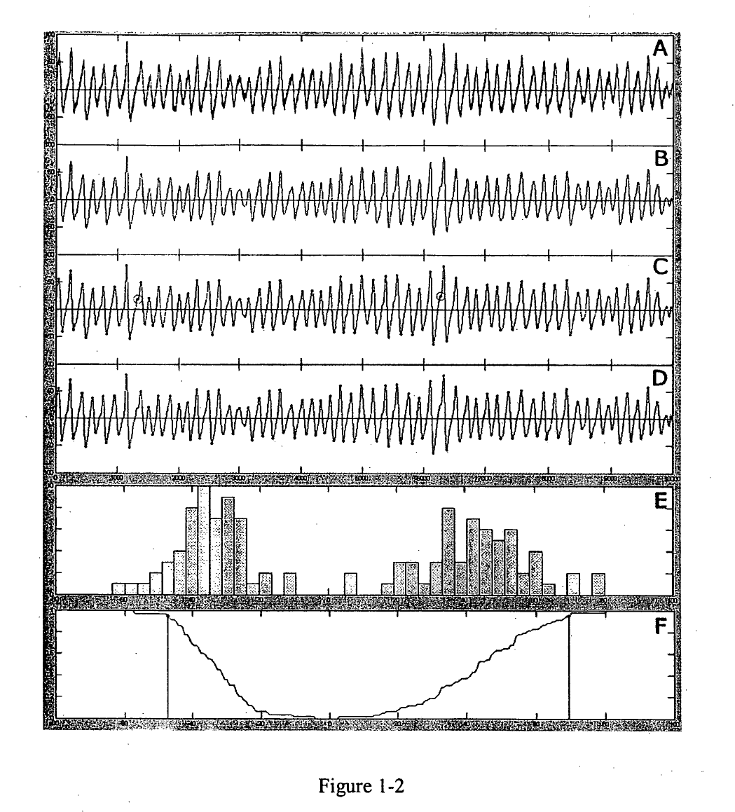

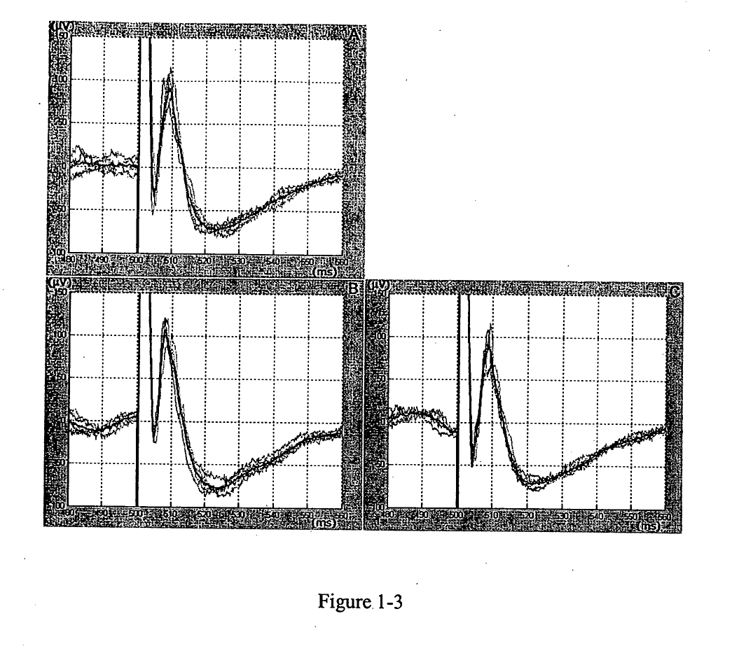

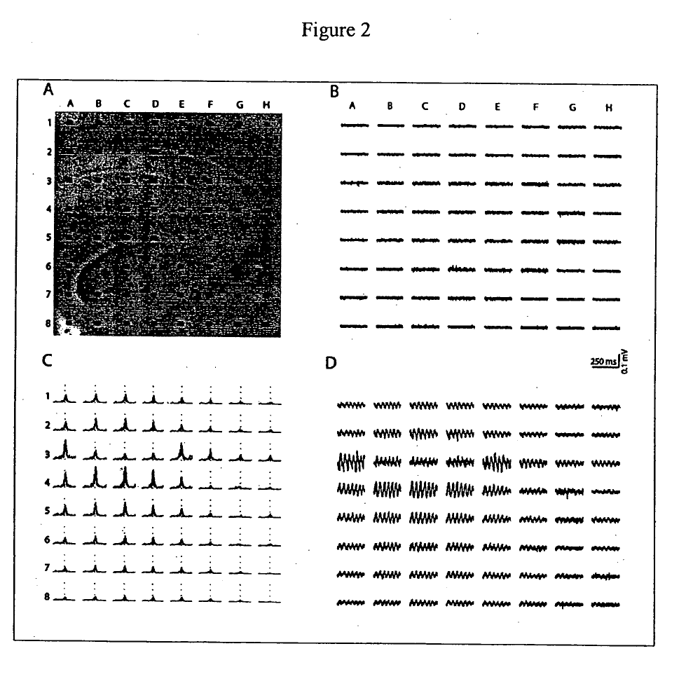

More specifically, spontaneous oscillations (EEG waves) from the neuronal tissue sample are measured by the MED (captured signal). Electrical noise from the equipment and the environment is then filtered. Isolating each basic wave and filtering the noise is typically accomplished by computing the power spectrum (energy as a function of frequency) using Fourier Transform analysis. An analysis of the power spectrum quantifies the energy content of the different EEG waves. Each wave can then be isolated by applying a narrow band filter to the Fourier transformation and then inverting it to obtain the time representation of the oscillation. The software included in the stimulation control system is then usually programmed to identify and isolate the independent rhythms, measure their relevant parameters, and update the information as the experiment progresses in order that the most current data is used when delivering an electrical pulse or train of pulses in phase with a particular rhythm. FIG. 1-2(A) is an example of a captured signal with a single wave having some amount of noise, while FIG. 1-2(B) shows the result of applying a high frequency filter to highlight the spontaneous oscillatory wave.

As illustrated in FIG. 1-2(C), the potential maximums and minimums of the filtered waveform are then computed using an approximation of the first derivative of the filtered waveform. The stochastic nature of the response can create “false” maximums and minimums, i.e., local maximums or minimums representing a plateau and not a true maximum. Two such “false” maximums, which are encircled, are also shown in FIG. 1-2(C). Using an approximation of the second derivative of the original filtered waveform, it is then possible to identify “false” maximum values as inflexion points, and to properly eliminate them. FIG. 1-2(D) shows the true maximums and minimums after this elimination process has been applied.

As seen in FIG. 1-2(E), the true maximums and minimums may then be used to construct two histograms, one for maximum values and the other for minimum values. From the histograms, it is possible to obtain both the positive and the negative cumulative probability distributions. These distributions represent the probability that an arbitrary maximum (alternatively, minimum) will have a magnitude that is greater (smaller) than a certain threshold voltage.

Once the threshold values have been computed, it is possible to deliver an electrical pulse or train of pulses either on the maximum peaks or the minimum peaks. For example, the software continuously captures spontaneous electrical waveforms from the tissue sample, analyzes them, and updates the relevant parameters. The stimulation control system user selects the particular stimulation pattern to execute and the particular stimulation site. In case more than one spontaneous oscillatory rhythm is present, the user may also select the particular rhythm to synchronize against, as well as the positive or negative phase for stimulation, and a probability threshold. From the probability level and the corresponding cumulative probability distribution, it is possible by inverting the latter to compute the expected maximum value that satisfies the selected probability threshold. Using the software-controlled output trigger facility present in many data acquisition cards, it is then possible to program delivery of the stimulation pattern to synchronize with the attainment of the calculated threshold voltage. Likewise, the recording of the evoked response can be started using the same triggering facility.

Thereafter, a candidate sample composition that may or may not contain a psychoactive compound is then contacted with the in vitro neuronal tissue sample. A network level electrical response, i.e., an array of extracellular voltages or potentials is then measured. We have found that a comparison of the network level electrical waveforms before and after the introduction of a stimulation pulse and/or the introduction of a psychoactive compound(s) provides information on the presence of and/or characterization of psychoactive compositions. More details on specific compounds will be provided below in the Examples.

One application providing significant information as to the presence of, or characterization of psychoactive compounds is the use of Fast Fourier Transforms (FFTs). FFT is used in a variety of disparate areas and is commonly used in a device known as a spectrum analyzer. The application of FFT to specific measurements in the measurement array and the comparison of that result to a specific measurement (at that same location prior to the introduction of the candidate composition) is instructive as to the presence, characterization, or pharmacological activity of a psychoactive compound. Specifically, if the candidate is psychoactive in the region of the neuronal sample that is analyzed, a comparison of the so-analyzed signals may show a shift in peak frequencies, amplitudes, a combination of the two, or other physiological effects.

Current Source Density (CSD) analysis is another useful application. A discussion of this analytical procedure is found, e.g., in Nicholson et al., “Theory of Current Source Density Analysis and Determination of Conductivity Tensor for Anuran Cerebellum,” Journal of Neurophysiology 38(2):356-68 (1975). This analytical procedure may be used to convert the potentials or voltages measured by the devices described herein, and convert them into a similar configured array of current flows and, more importantly, current magnitudes. By correlating the magnitude and direction of the currents as a function of time, current “sinks” and “sources” may be observed. The locations of such “sinks” and “sources” are instructive in determining the presence, characterization, or pharmacological activity of psychoactive drugs added to the in vitro neuronal sample.

In general, and unless the context shows another meaning for the term, when we use the term “characterization” or “characterizing” in referring to a psychoactive compound or composition, we mean that we have employed a series of observational measurement and analytical steps described here to produce a dataset having a particular form or format that allows subsequent practical, medical, or functional aspects of the compound or composition, e.g., chemical identification of, specific psychoactive activity of, suspected and/or resulting psychological ramifications of, or the like, of that compound or composition. Once the observed data are analyzed using the described procedures, the form or format of the dataset is such that it may then be readily and accurately compared with corresponding data generated from in vitro neuronal tissue samples contacted with known psychoactive compounds and analyzed in the same way. Further, the characterization dataset from a specific psychoactive may be further analyzed and contrasted to or compared with data by other methodologies (e.g., non-in vitro assay generated data).

### EXAMPLES

The following Examples are provided to show that a psychoactive compound can be detected and characterized by measuring a network level response in an in vitro neuronal tissue sample. Those skilled in the art will recognize that while specific embodiments have been illustrated and described, various modifications and changes may be made without it departing from the spirit and scope of the invention.

### Example 1

### Preparation of Multi-Electrode Array

Procedures for the preparation of the recently introduced Multi-Electrode Dish (Panasonic: MED probe) are described by Oka et al., J Neurosci Methods 93: 61-67 (1999). The device has an array of 64 planar microelectrodes, each having a size of 50×50 μm, arranged in an 8 by 8 pattern. Probes used in the experiments described below were medium arrays with 300 μm interelectrode spacing (Panasonic: MED-P530AP).

For sufficient adhesion of the slice to the probe, the surface of the MED probe was treated with 0.1% polyethylenimine (Sigma: P-3143) in 25 mM borate buffer, pH 8.4, for 8 hours at room temperature. The probe surface was rinsed 3 times with sterile distilled water. The probe (chamber) was then filled with DMEM/F-12 mixed medium, containing 10% fetal bovine serum (GIBCO: 16141-079) and 10% horse serum (GIBCO: 16050-122), for at least 1 hour at 37° C. DMEM/F-12 mixed medium is a 1:1 mixture of Dulbecco's Modified Eagle's Medium and Ham's F-12 (GIBCO: D/F-12 medium, 12400-024), supplemented with N₂ supplement (GIBCO: 17502-014) and hydrocortisone (20 nM, Sigma, H0888).

### Example 2

### Preparation of Hippocampal Slices

A 17-25 day old Sprague-Dawley rat was sacrificed by decapitation after anesthesia using halothane (2-Bromo-2chloro-1,1,1-trifluoroethane, Sigma: B4388), and the whole brain was removed. The brain was immediately soaked in ice-cold, oxygenated preparation buffer of the following composition (in mM): 124 NaCl, 26 NaHCO₃, 10 glucose, 3 KCl, 1.25 NaH₂PO₄, 1.5 CaCl₂, 0.5 MgSO₄, for approximately 2 minutes. Appropriate portions of the brain were trimmed and placed on the ice-cold stage of a vibrating tissue slicer (Leica: VT-1000S). The stage was immediately filled with both oxygenated and frozen preparation buffers. The thickness of each tissue slice was 350 μm. Each slice was gently taken off the blade using a blunt-end pipette, and immediately soaked in oxygenated preparation buffer for at least 1 hour at room temperature. A slice was then placed on the center of the MED probe, positioned to cover the 8×8 array. After positioning the slice, the MED probe was placed directly in a box filled with 95% O₂ and 5% CO₂ and allowed to recover and adhere at 32° C. for 1 hour.

### Example 3

### Electrophysiological Recording

During electrophysiological recording, the slices on the MED probe were placed in a small CO₂ incubator (Yamato: model IC400) at 32° C. The slices remained on the MED probe, which was attached to a recording interface, and a moisturized 95% O₂ and 5% CO₂ gas mixture was blown from above. In this condition, responses were recorded for more than 2 hours.

Carbachol was obtained from Sigma. A carbachol solution was applied to the neuronal samples at known concentrations of either 25 or 50 μM. The lower concentration was used in all except two cases. In these two cases, the concentration was increased to 50 μM after 25 μM was found to be insufficient to induce powerful oscillations. Carbachol solutions were prepared daily from frozen aliquots.

Spontaneous and evoked field potentials at all 64 sites were recorded simultaneously with the multi-channel recording system (Panasonic: MED64 system) at a 20 kHz sampling rate. In the case of the evoked response, one or more than two microelectrodes out of the 64 available were chosen for stimulating. Bipolar constant current pulses (20 to 40 μA, 0.1 msec) were produced by the data acquisition software through the isolator. The stimulating microelectrodes were selected by the 64 switch-box.

### Example 4

### Stimulation Control System

FIGS. 1-1 and 1-2 show a flowchart and algorithm of a Stimulation Control System that delivers a stimulation pattern of electrical pulses to a neuronal tissue slice in synchrony with the positive or negative peaks of oscillations. The oscillations may be spontaneous oscillations or oscillations themselves induced by an electrical pulse(s), chemical composition, or co-deposited neuronal tissue.

The effect of this Stimulation Control System is summarized in FIG. 1-3. First, a stimulation was delivered without using the system, and five consecutive responses were captured (FIG. 1-3A). Secondly, stimulation was delivered at the positive peak, and five consecutive responses were captured (FIG. 1-3B). Lastly, stimulation was delivered at the negative peak (trough), and five consecutive responses were captured (FIG. 1-3C).

When the stimulation was delivered at the positive peak, the amplitude of the first negative peak of the responses was about −20 uv. When the stimulation was delivered at the negative peak, the amplitude of the first negative peak of the responses was about −40 uv. There is also a difference in the second negative peak time between the two responses (524 ms vs. 520 ms). When the stimulation was delivered randomly, the responses were distributed between these two groups.

In the examples described hereinafter, the train of pulses was delivered at the peak of the oscillations; however, alternative pulse delivery patterns will be appreciated by one skilled in the art.

### Example 5

### Cholinergic Beta Rhythms

An example of cholinergically driven beta waves is shown in FIG. 2. Panel A shows the hippocampal slice placed upon the array of 64 electrodes. Panel B shows a control recording of background activity from the slice depicted in panel A. Some spontaneous single unit activity was observed, but there were no synchronized field potential oscillations. Panel C shows the Fast Fourier Transform computed for a two second sample taken from the slice depicted in panel A following induction of cholinergic rhythms. The dotted vertical line at 26 Hz indicates the frequency at which power was maximal for this particular time sample. Within-slice variations in rhythmic activity exist across trials, and in this particular slice, maximal power was approximately 11000±4000 mV² (mean±S.D.) within a range of 18-30 Hz. Panel D illustrates the field potential oscillations for the time sample corresponding to the power spectra in panel C. As shown in both panels C and D, power was greatest in the apical dendritic fields and directly on the pyramidal cell bodies of field CA1. As expected, the phase of the rhythms was reversed across the cell body layer, as exemplified in the traces from electrodes C2 and C4 in panel D.

### Example 6

### Laminar Profile and Regional Distribution of Sustained Network Response

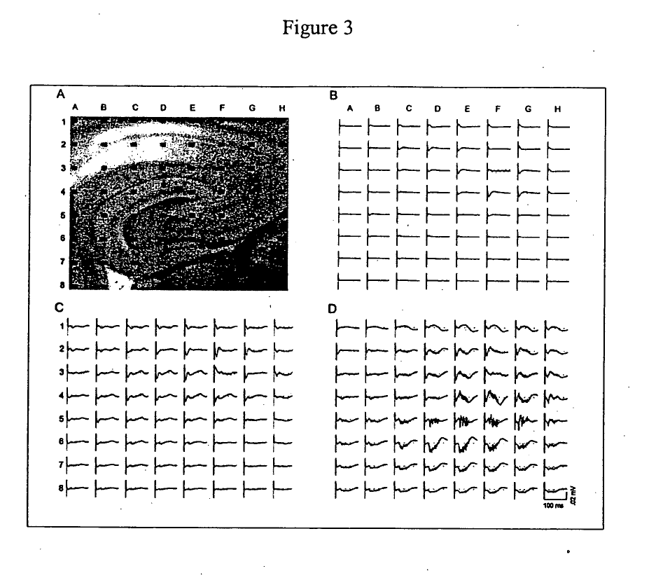

FIG. 3 compares a response in the absence of rhythmic activity to a network response evoked during cholinergically driven beta waves. As shown in Panel B, the dominant response in the control case is a rapidly developing and short lasting negative-going potential in the apical dendrites of field CA1 (e.g., electrode D2). This corresponds to the conventional field EPSP described in past physiological studies of the Schaffer-commissural (S-C) projections. Responses elsewhere in the slice are much smaller than those recorded in field CA1. With the addition of carbachol, two types of responses to single pulse stimulation emerge. In 16 of 53 recordings from eight slices, responses appear similar to the control responses described above (Panel C). However, in 37 of 53 traces from the same eight slices, carbachol produces two evident changes: 1) single pulses trigger bursts of spikes in the stratum pyramidale of field CA3; and 2) after-potentials become prominent features in both CA1 and CA3 (Panel D). An additional set of four slices was excluded from analyses because the delayed response was not observed in field CA1, most likely due to slight differences in slice preparation producing a weak CA3→CA1 projection.

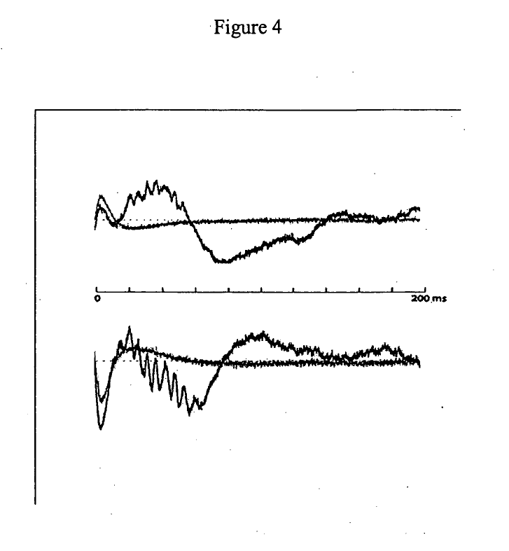

These effects can be seen more clearly in the single traces shown in FIGS. 3 and 4. The control response in field CA1, shown in gray, involved a typical biphasic response that reversed in polarity between stratum radiatum (the terminus of the stimulated fibers; FIG. 4, bottom) and stratum oriens (FIG. 4, top). The Schaffer-commissural response in field CA1 was greatly elaborated in the presence of carbachol (black trace, FIG. 4). A sequence composed of a slow negative followed by a slow positive potential was added to the short latency field EPSP recorded in the apical dendrites (e.g., electrode E3 in FIG. 2). The negative going component, which began about 10 msec after the initial response and 25 ms after stimulation, was accompanied by evidence of high frequency spiking. This was presumably a reflection of activity in the pyramidal cell bodies in field CA3 because the spikes were largest in that region. Presumably, the delayed response in the apical dendrites of CA1 was due to prolonged spiking of CA3 pyramidal cells reactivating the Schaffer commissural fibers, discussed in greater detail below. All components of the elaborated response in the apical dendritic terminal field of the Schaffer-commissural fibers reversed polarity in s. oriens (black trace, FIG. 4, top).

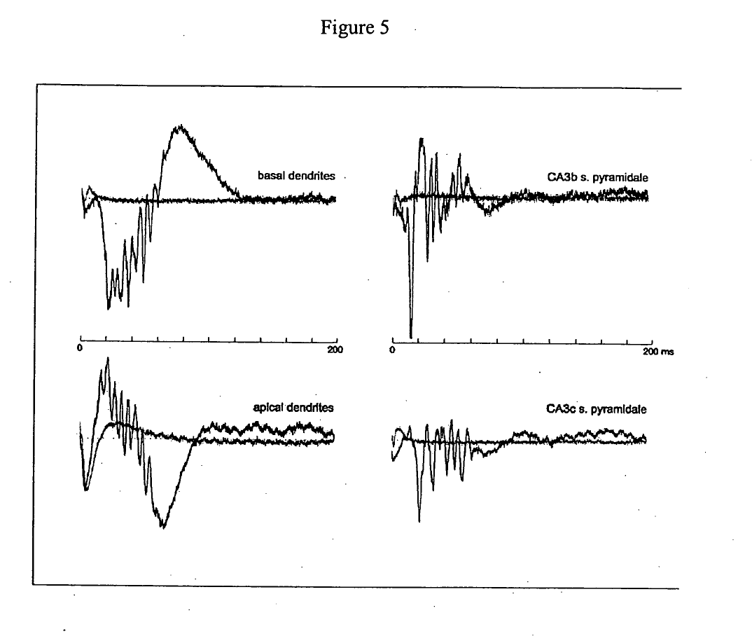

Carbachol also had very large effects on evoked potentials recorded in field CA3 (FIG. 5). The control response in the apical dendrites of CA3a was a typical field EPSP (FIG. 5, bottom left, gray trace) while the corresponding basal dendritic area, stratum oriens, showed the expected polarity reversal (FIG. 5, top left, gray trace). Addition of carbachol resulted in the appearance of a positive-going slow potential that began while the excitatory postsynaptic potential (EPSP) was still present and that was eventually replaced by a negative-going slow potential in the apical dendrites of CA3 (FIG. 5, bottom left, black trace). Note that the apical dendritic sequence of the slow potentials in CA3 (positive-negative) was opposite that in CA1. A phase-reversed version of the apical dendritic response occurred in the basal dendrites of CA3 (FIG. 5, top left, black trace). Bursts of spikes accompanied the first of the carbachol dependent slow potentials in CA3 (FIG. 5, right panels, black traces); these were pronounced in the cell body layer and became reduced in amplitude with distance from that layer. The bursts had a frequency of 100-200 Hz and began in the earliest phase of the first of post-EPSP slow waves. Since bursts of cell firing must be accompanied by nearby depolarizing currents, the negative-going waveform in CA3 basal dendrites corresponding to the cell spiking was most likely a depolarizing waveform (verified with current source density analysis, below) driving the CA3 cells to fire repetitively. Prolonged firing of CA3 pyramidal cells presumably caused a secondary activation of S-C fibers, resulting in the delayed, slow, negative-going potential recorded in CA1s. radiatum. Note that there was no cell spiking in the stratum pyramidale of CA3 in the control condition (FIG. 5, right panels, gray traces).

While detectable responses were virtually absent in fields CA3b and CA3c in the control condition, responses reverberated throughout the entirety of CA3 in the presence of cholinergic beta oscillations. Cell spiking was observed across the entirety of CA3 stratum pyramidale during an elaborated carbachol response (FIG. 5, right panels, black traces). Not only were network responses more spatially distributed control responses, they extended across a much longer time period. This topic will be addressed in greater detail below.

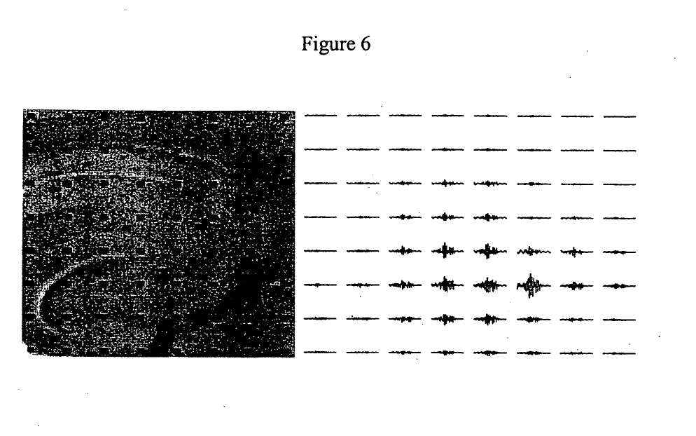

The records shown in FIGS. 3 and 4 suggest that the slow responses driven by S-C stimulation during cholinergic activation build up in field CA3 and then propagate into field CA1. Tests of this were carried out using the bursts of cell spikes described earlier. High-pass filtering the responses at 100 Hz to remove slow synaptic potentials revealed that the bursts were larger in field CA3 than in field CA1 (FIG. 6). Cross-correlations showed that the spikes in CA1 and CA3 were well correlated when the former were delayed by 2-4 msec from the latter, a value that accords well with the known conduction velocity of the Schaffer-commissural projections from CA3 to CA1. These results confirm the idea that spiking bursts triggered by stimulation originate in field CA3 and then are propagated from there to CA1.

### Example 7

### Time Course of Response

The minimum negative-going potential of the fast component occurred in the apical dendrites of field CA3 on average at 5.5±0.7 msec and in CA1 apical dendrites at 5.3±1 msec (mean±S.D., n=37 stimulation trials from 8 slices). The minimum of the slow component of the CA3 basal dendritic negative-going potential occurred at 24±10.5 msec. In CA1, the minimum value of the slow, negative-going potential in the apical dendritic field occurred significantly later at 52.4±19 msec. (paired t-test, two tails, p<0.0001). The high degree of variance in the slow potentials was attributable to individual variations across slices. Also, the delayed potentials integrated multiple synaptic events, unlike the control monosynaptic EPSPs, and thereby introduced additional degrees of variation.

The peak positive-going potential in CA3 apical dendrites corresponding to the slow negative-going potential in CA3 basal dendrites occurred at 17±7.4 msec. This peak was probably both a source for the depolarizing currents in the basal dendrites and reflected hyperpolarizing currents generated by feedback interneurons. Its maximum occurred at a significantly shorter time after the response than did the minimum negative-going slow potential in the basal dendritic field of CA3 (paired t-test, two tails, p<0.001), indicating that the positive-going waveform was not merely a reversal of the basal dendritic waveform. Additionally, the time course of its peak was consistent with IPSPs generated by feedback interneurons in response to the primary fast response in the CA3 apical dendrites.

### Example 8

### Response Size

The minimum amplitude of the fast negative-going potential was −60±40 μV in the CA3 apical dendritic field and −70±50 μV in the CA1 apical dendritic field. Neither of these values was significantly correlated with the minimum amplitude of the slow negative-going potential in CA1 apical dendrites (−105±43 μV) nor the slow negative-going potential in CA3 basal dendrites (−180±88 μV). On average, the minimum amplitudes of the slow components were more negative than the fast component minimum amplitudes (paired t-test, two tails, p<0.01) for both CA3 and CA1. The average maximum amplitude of the large positive-going waveform in CA3 apical dendrites was 170±135 μV. None of the amplitude measures were found to be significantly correlated with the power of the oscillations.

### Example 9

### Current Source Density Analysis

The method of continuous, two-dimensional current source density employed has been previously described (Shimono et al., supra). The 2-dimensional array of electrodes allows for simultaneous estimation of current flows in any direction within the plane of the slice. The data was low-pass filtered at 100 Hz and spatially smoothed by a 3×3-weighted average kernel (0 ⅛ 0, ⅛ ½ ⅛, and 0 ⅛ 0). The result was convolved with a 3×3 Laplacian kernel to produce a discrete approximation of the second spatial derivative. A limitation of the technique is that only large spatial patterns, with radii≧one-half of the interelectrode distance, can be accurately resolved. Additionally, low-pass filtering removes high-frequency data, limiting the amount of fine detail that can be observed.

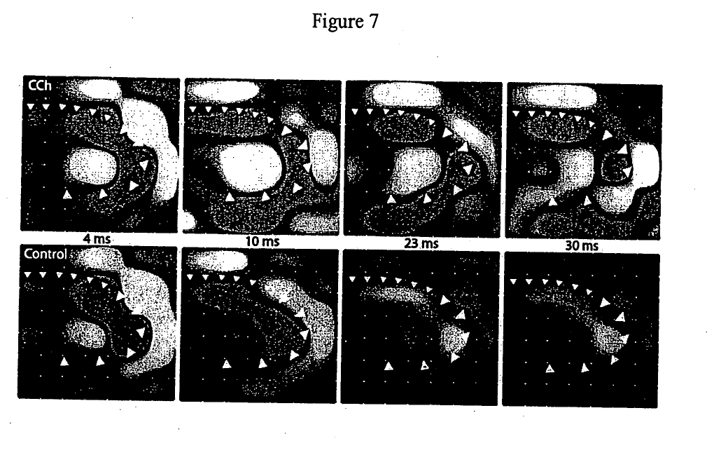

FIG. 7 shows the results of two-dimensional current source density analysis for selected time points in a 30 ms time window following stimulation to the S-C pathway. The estimated current sinks (gray) and sources (white) for the sustained network response in the presence of 25 μM carbachol are shown in the top four panels and for an evoked response in the absence of cholinergic activity in the bottom four panels. At 4 ms post-stimulation, the responses were quite similar, although stimulation in the presence of cholinergic rhythms evoked a response that extended across more of the network. In both cases, the major depolarizing current sinks occurred in the apical dendritic fields of CA1 and CA3. Corresponding current source dipoles were recorded across the cell body layers, but were not as prominent in the CA3 basal dendritic field for the carbachol response. Instead, it appeared that depolarizing currents were arising in the cell bodies of CA3 and beginning to produce a current sink. By 10 ms after stimulation, the entire network had fully mobilized in the carbachol case. The two major differences between the cholinergic response and the control response at this time were the appearance of a large current source in the CA3 apical dendrites and a well-formed current sink in the CA3 basal dendrites during the carbachol response. In the control condition, the dipoles in CA3 were the same as in CA1 (apical sinks/basal sources), whereas the dipole relationships reversed in CA3 when carbachol was present (CA3 apical source/basal sink, CA1 apical sink/basal source). It is also evident that the spread of activity was far greater when carbachol was present than that seen without carbachol, particularly in field CA3 where the activity reached to the terminus of the pyramidal cell layer (i.e., field Ca3c). Certainly related to greater spread, the intensity of activity was markedly increased in the presence of carbachol. At 23 ms following stimulation, the excitatory components of the evoked response were absent in the control condition, replaced by hyperpolarizing current sources in the apical dendritic fields of CA1 and CA3a. In contrast, the cholinergic response exhibited sustained excitatory current sinks in the apical dendrites of field CA1. Current sinks were also observed in the basal dendrites and cell bodies of CA3 at this time. At 30 ms post-stimulation, the sustained apical current sink and its corresponding basal source in CA1 remained robust in the carbachol response, while only weak apical current sources lingered in the control condition.

Current Source Density analyses yielded consistent patterns in 7 of the 8 slices at time points approximately 5, 10, 20, and 30 ms following stimulation. After 30 ms, a large degree of temporal variability in the estimated currents developed across slices, probably due to timing variations in the slow potentials and the emergence of prominent high-frequency components reflecting cell spiking in CA3. Thus, simultaneous current source densities computed after 30 ms post stimulation are not discussed here.

### Example 10

### Response Variability

Stimulation to the Schaffer collateral system in the presence of cholinergic rhythms elicited a sustained network response in approximately 70% of the stimulation trials, whereas in the other stimulation trials a response similar to a control EPSP was evoked. Analyses indicated that the phase of the oscillations (on which the stimulation pulse was delivered) was responsible for this variability. Analysis revealed that in 34 out of 47 stimulation trials in which the network response was evoked, 18 stimulation pulses clearly landed on a local minimum in the basal dendritic field of CA3. In 11 stimulation trials in which the network response occurred, stimulation arrived when potentials in the CA3 basal dendrites were close to zero. Only two out of 13 recordings in which a sustained response was not generated in the presence of carbachol showed the stimulation pulse arriving on a local minimum in the basal dendrites.

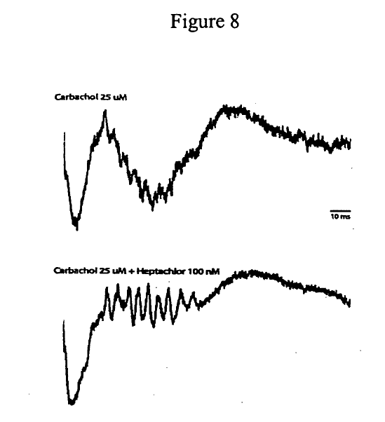

Network measures are not only more sensitive to subtle differences between closely related compounds, they are also more sensitive to extremely low concentrations. FIG. 8 shows an example of the effect of heptachlor, a cognition-impairing pesticide, at a concentration close to dangerous environmental levels, on Schaffer commissural stimulation. As shown, the slow component of the network complex evoked response described above is changed dramatically in the presence of 100 nM heptachlor, although the fast component, corresponding to the traditional evoked response, is not changed. Interestingly, the exact change observed is a complete elimination of the depolarizing phase of the slow component of the waveform. Elimination of this sustained period of activation leads to a prediction that cognitive function will be impaired. Cognitive effects of heptachlor exposure include memory deficits and confusion.

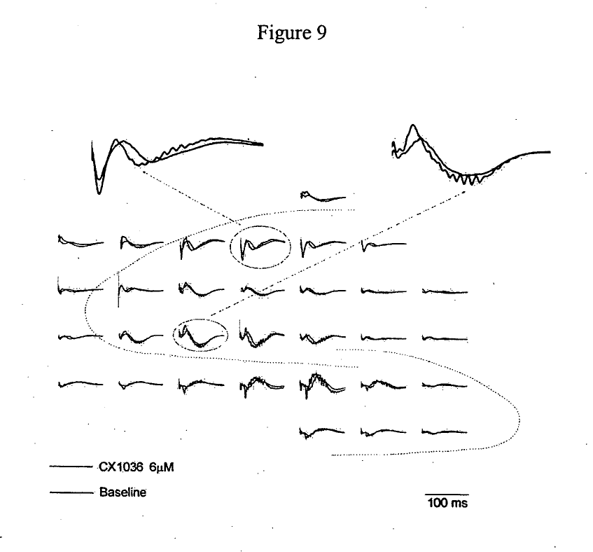

FIGS. 9-11 demonstrate that compounds from a cognition-enhancing pharmaceutical class induce varied effects at low concentrations. All compounds tested in this class enhanced both cell firing and excitatory potentials, indicating that these drugs work by facilitating excitatory transmission. However, there were subtle timing differences between the effects of the individual compounds.

FIG. 9 illustrates the network evoked response across the hippocampal network in the presence (gray) and absence (black) of CX1036, one of the cognition-enhancing agents tested. The characteristic increase in cell spiking and negative-going potentials is apparent; additionally, this particular compound was found to accelerate the time of onset for the slow potential.

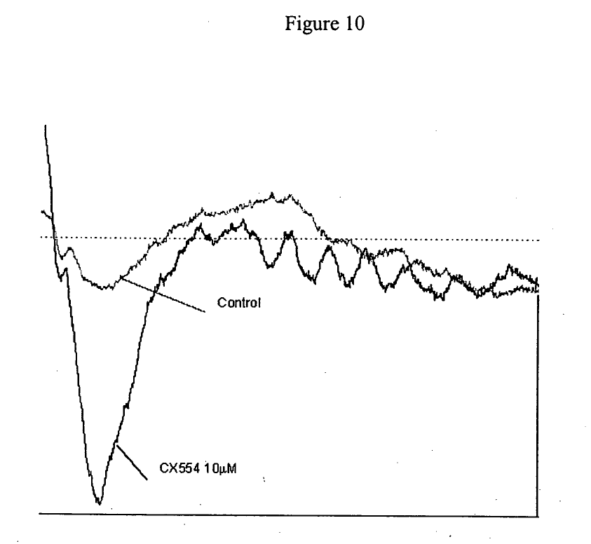

FIG. 10 shows the effect of another cognitive enhancer on the network evoked response in CA3 stratum pyramidale. As was the case with the other compounds in this class, cell spiking and excitatory potentials were enhanced; however, response timing was not affected by this compound.

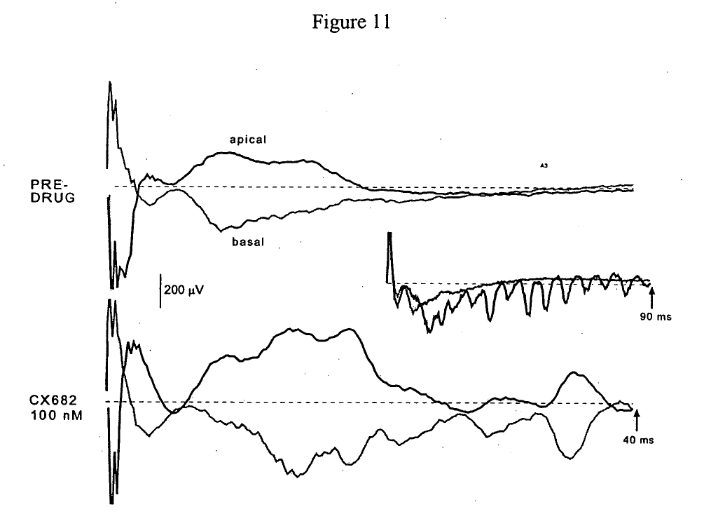

FIG. 11 depicts effects on the newtork evoked response in CA3 apical and basal dendritic fields of the last cognition-enhancing compound tested at a concentration that is two orders of magnitude lower than threshold for a monosynaptic response. It is again clear that drugs in this class increase cell firing and amplify excitatory potentials. Yet, in this case, the onset of the slow potential was found to be delayed.

These subtle differences in response timing between the closely-related compounds suggest that each of these drugs may be uniquely well-suited for treating a different cognitive deficit. It is interesting to note that the effects of these three drugs on monosynaptic responses are indistinguishable from one another.

Data Analysis

The data provided in the Examples suggest that rhythmic activity can have a profound affect on the responses generated by excitatory stimulation and, in addition, provide information as to the nature of such interactions. Responses to stimulation of the Schaffer-commissural projections, the principal associational system of hippocampus, became much more complex than the standard field EPSP following the introduction of a cholinergic agonist.

The magnitude and duration of the response to S-C stimulation varied greatly across trials following the initiation of rhythmic activity—with much of the variability attributable to the timing of stimulation pulses with respect to the rhythm phase. Thus, in the brain, synchronization associated with rhythmic activity may be a strategy for creating reliable and predictable time windows within which afferents arriving at opportune moments can exploit the processing capabilities of entire neuronal networks.

These results suggest that some degree of synchronization would be needed between rhythmic activity and excitatory inputs. The superficial layers of entorhinal cortex are the primary hippocampal afferent and are reported to generate beta waves when infused with cholinergic agonists (Shimono et al., supra). Interestingly, the deep layers of entorhinal cortex, an important target of hippocampus and subiculum, produce gamma rhythms under the same conditions (van der Linden et al., J Neurophysiol 82(5): 2441-2450 (1999)). lijima and colleagues reported that the transfer of information from the entorhinal cortex to the hippocampus was frequency-dependent, proposing that this frequency-dependent transfer may be involved in selectively gating the entry of information into the hippocampus (Iijima et al., Science 272: 1176-1179 (1996)). Similarly, gamma activity has been reported to occur in hippocampus following carbachol infusion (Fisahn et al., supra; Fellous et al., supra), and gamma coherence between hippocampus and entorhinal cortex is relatively high in vivo (Charpak et al., Eur J Neurosci 7: 1548-1557 (1995); Chrobak et al., J Neurosci 18: 388-398 (1998)). In any event, hippocampus and retrohippocampal cortex appear to have sufficiently similar local circuits so that the cholinergic septal projections can provide the synchronization required by the beta activity.

Cortical rhythms are usually thought to synchronize afferents and thereby allow them to be more effective than would be the case if they arrived in temporally scattered manner. Other studies have shown that certain naturally occurring rhythms have deep relationships with synaptic plasticity. The present results add to the list of potential functions by showing that rhythmic activity presents windows of opportunity for afferents such that properly timed arrival of a modest input can result in a large and temporally extended response. Such responses, and the characterization of such, provide a powerful detection and characterization means for centrally active agents.

All publications and patent applications cited in this application are herein incorporated by reference in their entirety. Although the foregoing invention has been described by way of illustration and example for purposes of clarity and understanding, it will be readily apparent to those of ordinary skill in the art in light of the teachings of this invention that certain changes and modifications may be made thereto without departing from the spirit or scope of the appended claims.

### REFERENCES

Behrends J C, ten Bruggencate G (1993). Cholinergic modulation of synaptic inhibition in the guinea pig hippocampus in vitro: excitation of GABAergic interneurons and inhibition of GABA-release. J Neurophysiol 69: 626-629.
Charpak S, Pare D, Llinas R (1995). The entorhinal cortex entrains fast CA1 hippocampal oscillations in the anaesthetized guinea-pig: role of the monosynaptic component of the perforant path. Eur J Neurosci 7: 1548-1557.
Chrobak J J, Buzsaki G (1998). Gamma oscillations in the entorhinal cortex of the freely behaving rat. J Neurosci 18: 388-398.
Creager R, Dunwiddie T, Lynch G (1980). Paired pulse and frequency facilitation in the CA1 region of the in vitro rat hippocampus. J Physiol (London) 299: 409-424.
Fellous J M, Sejnowski T J (2000). Cholinergic induction of oscillations in the hippocampal slice in the slow (0.5-2 Hz), theta (5-12 Hz), and gamma (35-70 Hz) bands. Hippocampus 10: 187-197.
Fisahn A, Pike F G, Buhl E H, Paulsen O (1998). Cholinergic induction of network oscillations at 40 Hz in the hippocampus in vitro. Nature 394: 186-189.
Hájos N, Papp E C, Acsády L, Levey A I, Freund T F (1998). Distinct intemeuron types express m2 muscarinic receptor immunoreactivity on their dendrites or axon terminals in the hippocampus. Neuroscience 82: 355-376.
Huerta P T, Lisman J E (1993). Heightened synaptic plasticity of hippocampal CA1 neurons during a cholinergically induced rhythmic state. Nature 364: 723-725.
Iijima T, Witter M P, Ichikawa M, Tominaga T, Kajiwara R, Matsumoto G (1996). Entorhinal-hippocampal interactions revealed by real-time imaging. Science 272: 1176-1179.
Konopacki J, MacIver M B, Bland B H, Roth S H (1987). Carbachol-induced EEG ‘theta’ activity in hippocampal brain slice. Brain Res 405: 196-198.
Levey A I, Edmunds S M, Koliatsos V, Wiley R G, Helman C J (1995). Expression of m1-m4 muscarinic acetylcholine receptor proteins in rat hippocampus and regulation by cholinergic innervation. J Neurosci 15: 4077-4092.
Madison D V, Lancaster B, Nicoll R A (1987). Voltage-clamp analysis of cholinergic action in the hippocampus. J Neurosci 7: 733-741.
Nakajima Y, Nakajima S, Leonard R J, Yamaguchi K (1986). Acetylcholine raises excitability by inhibiting the fast transient potassium current in cultured hippocampal neurons. Proc Natl Acad Sci USA 83: 3022-3026.
Oka H, Shimono K, Ogawa R, Sugihara H, Taketani M (1999). A new planar multielectrode array for extracellular recording: application to hippocampal acute slice. J Neurosci Methods 93: 61-67.
Shimono K, Brucher F, Granger R, Lynch G, Taketani M (2000). Origins and distribution of cholinergically induced beta rhythms in hippocampal slices. J Neurosci 20: 8462-8473.
van der Linden S, Panzica F, de Curtis M (1999). Carbachol induces fast oscillations in the medial but not in the lateral entorhinal cortex of the isolated guinea pig brain. J Neurophysiol 82(5): 2441-2450.
Vertes R P, Kocsis B (1997). Brainstem-diencephalo-septohippocampal systems controlling the theta rhythm of the hippocampus. Neuroscience 81: 893-926.
Williams J H, Kauer J A (1997). Properties of carbachol-induced oscillatory activity in rat hippocampus. J Neurophysiol 78: 2631-2640.

1: A process for the detection of a psychoactive compound in an in vitro neuronal tissue sample comprising:
a) measuring at least one spontaneous oscillation or at least one induced oscillation from said in vitro neuronal tissue sample;
b) comparing a network level electrical response of said in vitro neuronal tissue sample contacted with a candidate sample composition with a network level electrical baseline to determine a difference between said network level electrical response and said network level electrical baseline; and
c) detecting the presence or absence of the psychoactive compound in said candidate sample composition based upon the difference between said network level electrical response and said network level electrical baseline.

2: The process of claim 1 further comprising the step of characterizing said psychoactive compound by comparing the difference between said network level electrical response and said network level electrical baseline.

3: The process of claim 1 further comprising the step of delivering at least one timed electrical pulse to said in vitro neuronal tissue sample.

4: The process of claim 1 wherein said induced oscillation is generated by introduction of a chemical composition.

5: The process of claim 4 wherein said chemical composition mimics the actions of acetylcholine, serotonin, or a catecholamine.

6: The process of claim 5 wherein said chemical composition comprises carbachol.

7: The process of claim 4 wherein said chemical composition is a stimulating composition.

8: The process of claim 4 wherein said induced oscillation is generated by electrical stimulation.

9: The process of claim 4 wherein said oscillation is generated by co-deposited neuronal tissue.

10: The process of claim 1 further comprising a rendering step of applying Fast Fourier Transform analysis to said at least one spontaneous or induced oscillation.

11: The process of claim 1 further comprising a rendering step of applying Current Source Density analysis to at least one spontaneous or induced oscillation.

12-18. (canceled)

19: A method for the detection and characterization of a psychoactive compound in an in vitro neuronal tissue sample comprising the steps of:
a) measuring at least one spontaneous oscillation generated by said in vitro neuronal tissue sample;
b) measuring a baseline network level electrical response in the in vitro neuronal tissue sample;
c) contacting the in vitro neuronal tissue sample with a candidate sample composition;
d) measuring a resulting network level electrical response in the in vitro neuronal tissue sample; and
e) comparing the resulting network level electrical response with the baseline network level electrical response to detect the presence or absence of said psychoactive compound in said candidate sample composition.

20: The method of claim 19 further comprising a rendering step of applying Fast Fourier Transform analysis to said at least one spontaneous oscillation.

21: The method of claim 20 wherein said rendering step is performed by a predictive software.

22: The method of claim 19 further comprising a rendering step of applying Current Source Density analysis to said at least one spontaneous oscillation.

23: The method of claim 22 wherein said rendering step is performed by a predictive software.

24: The method of claim 19 further comprising the step of delivering at least one timed electrical pulse to said in vitro neuronal tissue sample.

25: The method of claim 24 wherein said timed electrical pulse is delivered at a specific point in said spontaneous oscillation.

26: The method of claim 25 wherein said specific point is determined by a predictive software.

27: The method of claim 25 wherein said specific point is a peak of said spontaneous oscillation.

28: The method of claim 19 further comprising the step of adding a chemical composition to said in vitro neuronal tissue sample prior to detection of said baseline network level electrical response.

29: The method of claim 28 wherein said chemical composition mimics the actions of acetylcholine, serotonin, or a catecholamine.

30: The method of claim 29 wherein said chemical composition comprises a cholinomimetic compound.

31: The method of claim 30 wherein said chemical composition comprises carbachol.

32: The method of claim 19 further comprising the step of co-depositing neuronal tissue with said in vitro neuronal tissue sample prior to detection of said baseline network level electrical response.

33: The method of claim 19 further comprising the step of delivering electrical stimulation to said in vitro neuronal tissue sample prior to detection of said baseline network level electrical response.

34: The method of claim 19 wherein said baseline network level electrical response and said resulting network level electrical response are selected from the group consisting of theta, beta, and gamma EEG waves.

35-43. (canceled)
